(19) United States
(12) Patent Application Publication
(10) Pub. No.: US 2019/0192054 A1
(43) Pub. Date: Jun. 27, 2019

(54) PEG FOR REHABILITATION

(71) Applicants: NIHON KOHDEN CORPORATION, Tokyo (JP); KEIO UNIVERSITY, Tokyo (JP)

(72) Inventors: Junichi USHIBA, Yokohama-shi, Kanagawa (JP); Shohei TSUCHIMOTO, Yokohama-shi, Kanagawa (JP); Meigen LIU, Shinjuku-ku, Tokyo (JP); Michiyuki KAWAKAMI, Shinjuku-ku, Tokyo (JP); Tatsuo YOSHIDA, Shinjuku-ku, Tokyo (JP); Yoshinobu ONO, Shinjuku-ku, Tokyo (JP); Yoshiyuki FUJITA, Shinjuku-ku, Tokyo (JP); Kaoru IMAJO, Shinjuku-ku, Tokyo (JP); Fumihiko MURAYAMA, Shinjuku-ku, Tokyo (JP)

(73) Assignees: NIHON KOHDEN CORPORATION, Tokyo (JP); KEIO UNIVERSITY, Tokyo (JP)

(21) Appl. No.: 16/328,483

(22) PCT Filed: Aug. 21, 2017
(86) PCT No.: PCT/JP2017/029790

§ 371 Date: Feb. 26, 2019

(30) Foreign Application Priority Data
Aug. 31, 2016 (JP) 2016-169545

(51) Int. Cl. A61B 5/11; A61B 5/00; A63B 24/00; A61B 5/22; A61B 5/16; A61B 5/103

(52) U.S. Cl. CPC A61B 5/1125; A61B 5/486; A63B 24/0003; A61B 5/225; A61B 5/16; A61B 5/1036; A61B 5/0004; A61B 5/7455; A61B 2562/0247; A61B 2505/09; A63B 2024/0053; A61B 5/6896; A61B 5/7475

(57) ABSTRACT

A peg (3) is placed on a peg board (2) by a subject. A detection unit (32) detects a contact state between a peg body (31) and a body portion of the subject. A signal transmission unit (33) transmits a signal indicative of a detection result from the detection unit (32).

### TECHNICAL FIELD

The present invention relates to a peg to be placed on a peg board by a subject in rehabilitation performed for recovery of a finger function of the subject.

### BACKGROUND

The peg is a bar-like member having a predetermined shape and color. A hole corresponding to the shape and color of the peg is formed in the peg board. The subject grips the peg with his/her hand or fingertip and inserts it into the hole on the designated peg board.

Japanese Patent Publication No. 2010-284293A discloses a system for quantitatively evaluating the result of rehabilitation using a peg and a peg board in this way.

### SUMMARY OF INVENTION

### Technical Problem

The evaluation in the system described in Japanese Patent Publication No. 2010-284293A is merely based on whether or not the peg is inserted in the hole of the peg board. In order to accurately grasp the degree of functional recovery of a subject, that is, the progress state of rehabilitation, it is desired that more detailed evaluation is quantitatively and objectively performed.

Therefore, it is required to evaluate the progress state of rehabilitation in more detail and objectively.

### Solution to Problem

According to one aspect of the disclosure, there is provided a peg to be placed on a peg board by a subject, comprising:

a peg body;

a detection unit configured to detect a contact state between the peg body and a body portion of the subject; and

a signal transmission unit configured to transmit a signal indicative of a detection result from the detection unit.

According to such a configuration, not only the result of simply determining whether or not the peg can be moved to a predetermined position, but also the following information can be acquired:
the timing when the finger of the subject comes into contact with the peg (the initial timing of the peg moving operation);
the timing when the finger of the subject is separated from the peg (the termination or failure timing of the peg moving operation); and
the time period during which the finger of the subject is in contact with the peg (the duration of the peg moving operation).
   Therefore, it is possible to objectively (quantitatively) evaluate the progress status of rehabilitation in more detail than the conventional way.

### BRIEF DESCRIPTION OF DRAWINGS

FIG. 1 schematically illustrates a basic structure of a peg according to one embodiment.

FIG. 2 schematically illustrates a first modified example of the peg.

FIG. 3A schematically illustrates a second modified example of the peg.

FIG. 3B schematically illustrates a third modified example of the peg.

### DESCRIPTION OF EMBODIMENTS

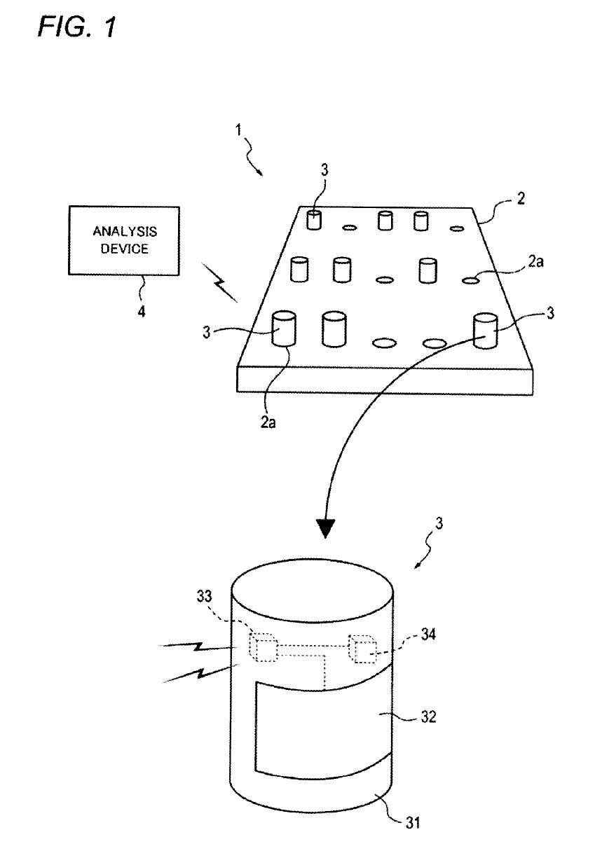

Examples of the embodiment of the invention will be described below in detail with reference to the accompanying drawings. FIG. 1 schematically illustrates a rehabilitation support system 1 (hereinafter abbreviated as support system 1) according to one embodiment. The support system 1 comprises a peg board 2, at least one peg 3, and an analysis device 4.

The peg board 2 has a plurality of holes 2a. A plurality of pegs 3 are inserted into some of the plurality of holes 2a. The plurality of pegs 3 may include plural types. The type of the peg 3 is distinguished by shape, size, color, and the like. Different pegs can be used appropriately, for example, by changing the easiness of holding in accordance with the progress of rehabilitation. In the present embodiment, each peg 3 is provided with a peg body 31 having a columnar appearance. On the other hand, the plurality of holes 2a include a plurality of types so as to correspond to the type of the peg 3. That is, the type of the hole 2a is distinguished by shape, size, color, and the like.

For example, a subject pulls the designated peg 3 out of the hole 2a and inserts it into another designated hole 2a according to the instructions of the medical staff.

In order to increase variations of the work according to the progress of the rehabilitation, the peg 3 locating at a place other than the hole 2a may be inserted into the designated hole 2a.

The peg 3 includes a detection unit 32 and a signal transmission unit 33. The detection unit 32 is configured to detect the contact state between the finger of the subject (an example of a part of the subject's body) and the peg body 31. The signal transmission unit 33 is configured to transmit a signal indicating the detection result from the detection unit 32 to the analysis device 4. In the present embodiment, the signal transmission from the signal transmission unit 33 to the analysis device 4 is performed using a well-known wireless communication technique.

The analysis device 4 is configured to acquire information on the contact state of the subject's finger with respect to the peg 3 based on the signal received from the signal transmission unit 33 of the peg 3.

According to such a configuration, not only the result of simply determining whether or not the peg 3 can be moved to a predetermined position, but also the following information can be acquired:
the timing when the finger of the subject comes into contact with the peg 3 (the initial timing of the peg moving operation);
the timing when the finger of the subject is separated from the peg 3 (the termination or failure timing of the peg moving operation); and
the time period during which the finger of the subject is in contact with the peg 3 (the duration of the peg moving operation).

Therefore, it is possible to objectively (quantitatively) evaluate the progress status of rehabilitation in more detail than the conventional way.

The detection unit 32 may be configured to particularly detect at least one of the contact strength and the contact area between the finger of the subject and the peg body 31.

In the case of detecting the contact strength, it is possible to know how much force the subject holds the peg 3.

The force to hold the peg 3 can be one of the indices for knowing the recovery degree of the finger function. In the case of detecting the contact area, it is possible to know the number of fingers whose subject is holding the peg 3.

The number of fingers that can participate in the operation of holding the peg 3 can also be one of the indices for knowing the recovery degree of the finger function. Therefore, the progress status of rehabilitation can be objectively (quantitatively) evaluated with further details.

In the present embodiment, a sheet-like coil is disposed on the outer peripheral face of the peg body 31 as the detection unit 32. The signal transmission unit 33 is configured to transmit a signal corresponding to the value of the inductance of the coil to the analysis device 4.

In this case, since the inductance changes when the finger of the subject touches the coil, the presence or absence of contact can be determined in the analysis device 4.

Further, since the inductance varies according to the contact area, the analysis device 4 can determine the number of fingers in contact based on the value of the inductance.

Same or similar information can be obtained by a capacitive sensor instead of the above coil.

Additionally or alternatively, the detection unit 32 may include at least one of a pressure-sensitive sensor and a touch key. The signal transmitting unit 33 is configured to transmit a signal corresponding to the output of at least one of the pressure-sensitive sensor and the touch key to the analysis device 4.

In this case, the analysis device 4 can determine the presence or absence of contact of the subject's finger with respect to the peg 3 based on the fact that the output of at least one of the pressure-sensitive sensor and the touch key has changed.

Further, the analysis device 4 can determine the contact strength of the subject's finger with respect to the peg 3 based on the magnitude of the value indicated by at least one of the pressure-sensitive sensor and the touch key.

As shown in FIG. 1, the peg 3 may comprise a motion sensor 34. The motion sensor 34 is configured to detect at least one of the position, posture, displacement, speed, and acceleration of the peg body 31. The motion sensor 34 may be constituted by at least one of an acceleration sensor, a geomagnetic sensor, a GPS sensor, and a gyro sensor. In this case, the signal transmission unit 33 is also configured to transmit a signal indicating the detection result of the movement sensor 34.

According to such a configuration, in addition to the information on the contact state between the finger of the subject and the peg 3, information on the movement of the peg 3 caused by the subject can also be acquired, so that the progress of the rehabilitation state can be evaluated. For example, in the analysis device 4, it is possible not only to determine the speed of moving the peg 3 to a predetermined place, but also to estimate the movement of a wrist or an arm of the subject based on the posture of or the route of the peg 3 to reach the predetermined place.

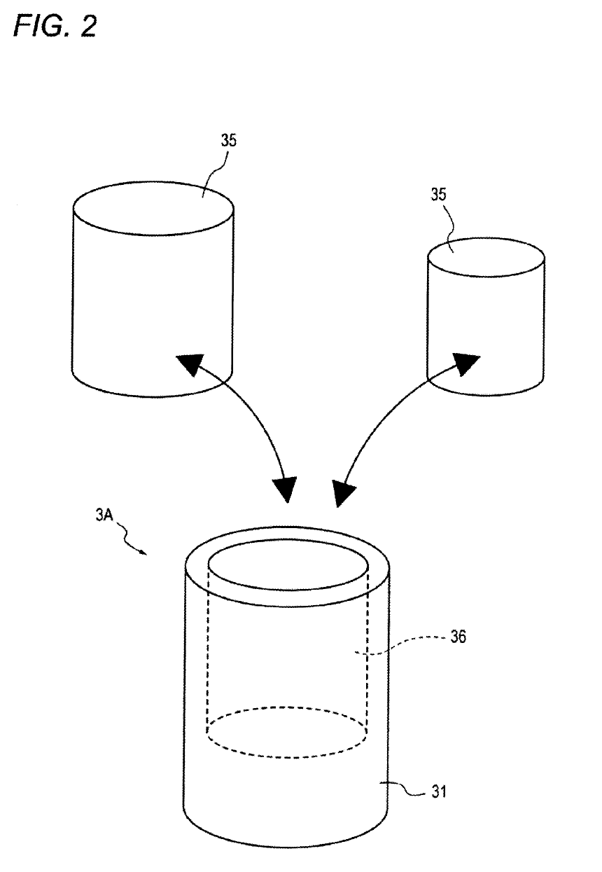

FIG. 2 schematically illustrates a peg 3A according to a first modified example. In addition to the configuration described with reference to FIG. 1 that is not shown in this figure, the peg 3A is provided with a housing portion 36 capable of housing a weight 35. The weight 35 is attachable to and detachable from the housing portion 36.

According to such a configuration, by housing the weights 35 having different weights in the housing portion 36, it is possible to change the weight of the peg 3A and to make the contents of rehabilitation have diversity. Since the detection unit 32 and the signal transmission unit 33 are provided, the unit price of the peg 3A can not help being higher than that of the conventional non-functional peg. However, when performing rehabilitation using pegs of different weights, it is not necessary to change the basic configuration of the peg 3A including the detection unit 32 and the signal transmission unit 33, but the weight of the weight 35 housed in the housing unit 36 may be just changed. In other words, versatility of the peg 3A for various rehabilitation can be improved.

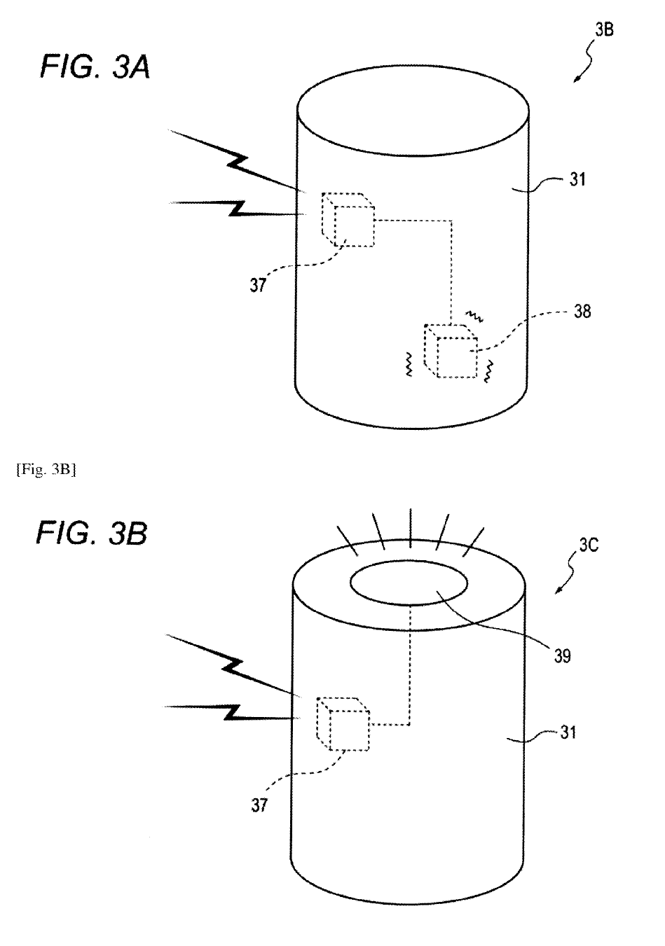

FIG. 3A schematically illustrates a peg 3B according to a second modified example. In addition to the configuration described with reference to FIG. 1 that is not shown in this figure, the peg 3B comprises a signal receiving unit 37 and a vibration generating unit 38.

The signal receiving unit 37 is configured to be able to receive a command signal from the analysis device 4 or another external device. In the present example, the command signal from the analysis device 4 or another external device is received through the well-known wireless communication technology.

The vibration generating unit 38 is configured to generate vibration in accordance with the command signal received by the signal receiving unit 37. The vibration generating unit 38 can be realized by an eccentric motor or the like.

With such a configuration, not only can the diversity of the content of rehabilitation be further enhanced but also the progress status of rehabilitation can be objectively evaluated with further details. For example, the medical staff issues an instruction such as “If you feel vibration, please release the peg”. Upon sensing the vibration generated by the vibration generating unit 38, the subject releases his/her hand from the peg 3B. As a result, it can be confirmed whether the sensory nerve of the subject functions properly.

FIG. 3B schematically illustrates a peg 3C according to a third modified example. In addition to the configuration described with reference to FIG. 1 that is not shown in this figure, the peg 3C comprises a signal receiving unit 37 and a light emitting unit 39. The function of the signal receiving unit 37 is the same as that described with reference to peg 3B.

The light emitting unit 39 is configured to generate vibration in accordance with the command signal received by the signal receiving unit 37. The light emitting section 39 can be realized by a bulb, a light emitting diode, an organic EL element, or the like.

With such a configuration, not only can the diversity of the content of rehabilitation be further enhanced but also the progress status of rehabilitation can be objectively evaluated with further details. For example, the medical staff issues an instruction such as “Please release the peg when it emits light”. When the light emitting unit 39 emits light, the subject releases his/her hand from the peg 3C. As a result, it can be confirmed whether the sensory nerve of the subject functions properly.

The above embodiment is exemplified to facilitate the understanding of the concept of the present disclosure without any limitation. The above exemplified embodiment may be modified or improved without departing from the concept of the present disclosure. Herein, the phrase “at least one of A, B and C” may be construed as an alternative expression that means one or more of A. B and C may be used.

In the above embodiment, the peg body 31 has a cylindrical appearance. However, depending on the purpose and stage of rehabilitation, the shape of the peg body 31 can be appropriately selected.

In the above embodiment, the signal transmission from the signal transmission unit 33 of the peg 3 to the analysis device 4 is performed by radio communication. However, the signal transmission from the signal transmission unit 33 of the peg 3 to the analysis device 4 may be performed by wired communication.

In the second modified example and the third modified example described above, the command signal is transmitted from the analysis device 4 or another external device to the signal receiving unit 37 of the peg 3 by wireless communication. However, the command signal from the analysis device 4 or another external device to the signal receiver 37 of the peg 3 may be transmitted by wired communication.

In the third modified example described above, the light emitting unit 39 is configured to emit light based on a command signal received by the signal receiving unit 37. However, the light emitting unit 39 may be configured to emit light independently of the command signal. For example, instead of changing the color of the peg body 31, by causing the light emitting section 39 to emit light in different colors, versatility of the peg 3C for various rehabilitations can be improved without changing the basic configuration.

The present application is based on Japanese Patent Application No. 2016-169545 filed on Aug. 31, 2016, the entire contents of which are hereby incorporated by reference.

1. A peg to be placed on a peg board by a subject, comprising:
a peg body;
a detection unit configured to detect a contact state between the peg body and a body portion of the subject; and
a signal transmission unit configured to transmit a signal indicative of a detection result from the detection unit.

2. The peg according to claim 1,
wherein the detection unit is configured to detect at least one of contact strength and a contact area between the peg body and the body portion of the subject.

3. The peg according to claim 2,
wherein the detection unit comprises at least one of a coil, a capacitive sensor, a touch key and a pressure-sensitive sensor that is disposed on a surface of the peg body.

4. The peg according to claim 1, further comprising:
a motion sensor configured to detect at least one of position, posture, displacement, speed and acceleration of the peg body,
wherein the signal transmission unit is configured to transmit a signal indicative of a detection result from the motion sensor.

5. The peg according to claim 1, further comprising:
a housing portion capable of housing a weight.

6. The peg according to claim 1, further comprising:
a signal receiving unit configured to receive a command signal from an external entity; and
a vibration generating unit configured to generate vibration in accordance with the command signal.

7. The peg according to claim 1, further comprising:
a light emitting unit.

8. The peg according to claim 3, further comprising:
a motion sensor configured to detect at least one of position, posture, displacement, speed and acceleration of the peg body, wherein the signal transmission unit is configured to transmit a signal indicative of a detection result from the motion sensor;
a housing portion capable of housing a weight;
a signal receiving unit configured to receive a command signal from an external entity;
a vibration generating unit configured to generate vibration in accordance with the command signal; and
a light emitting unit.
